(19) United States
(12) Patent Application Publication
(10) Pub. No.: US 2023/0299944 A1
(43) Pub. Date: Sep. 21, 2023

(54) Computer-implemented method for inputting and storing patient data

(71) Applicant: Katharina HEIL, Graz (AT)

(72) Inventor: Katharina HEIL, Graz (AT)

(21) Appl. No.: 18/015,772

(22) PCT Filed: Jul. 13, 2021
(86) PCT No.: PCT/EP2021/069452

§ 371 Date: Jan. 12, 2023

(30) Foreign Application Priority Data
Jul. 14, 2020 (EP) 20185651.5

(51) Int. Cl. H04L 9/08; G16H 10/60; H04L 9/00

(52) U.S. Cl. CPC H04L 9/0825; G16H 10/60; H04L 9/50

(57) ABSTRACT

A computer-implemented method for inputting and storing patient data in a computer system by multiple users, includes the following steps: defining a study key, defining one user key per user by means of the study key, allocating the user keys to the users, and inputting patient data by the individual users by means of the respective user key, wherein the inputted patient data are stored in the computer system in an encrypted manner by means of the user key.

### TECHNICAL FIELD

The invention relates to a computer-implemented method for inputting and storing patient data in a computer system by multiple users.

### BACKGROUND

In the computer-assisted processing of patients' medical data, there are particularly high requirements and particularly strict legal provisions concerning data protection. The patient data are generally personal data, for which a particularly high level of data security is to be ensured.

For studies in the medical field, it must be possible to collect and process such patient data also across many sites and also internationally. At the same time, comprehensive data are to be collected in order to ensure that such studies are properly documented and recorded.

Specifically in the case of the collection or inputting of such patient data within the context of medical studies, much action is still required in respect of the digitization of such processes.

### SUMMARY

The problem underlying the disclosure is to provide a computer-implemented method for inputting and storing patient data in a computer system by multiple users and an associated computer system, by means of which patient data can be inputted and stored across multiple sites with a particularly high level of security.

This problem is solved with a computer-implemented method for inputting and storing patient data in a computer system by multiple users, in which the following steps are carried out: firstly defining a study key, secondly defining one user key per user by means of the study key, thirdly allocating the user keys to the users, and fourthly inputting patient data by the individual users by means of the respective user key, wherein the inputted patient data are stored in the computer system in an encrypted manner by means of the user key. The term “study” is to be understood as meaning any type of study or investigation as well as other types of project with content-related data, that is to say not only medical or, for example, pharmaceutical studies.

The particular feature of the procedure of this type is that the encryption on inputting of the patient data is based on a two-step generation of keys, namely a study key for the medical study itself and a user key for each of the users, wherein each user key is additionally generated on the basis of the study key. The study key is thus processed or integrated in the user key, so that each user key also contains the information of the study key in an encrypted manner.

Owing to this particular system of keys, it is possible that the data inputted by each user are in principle encrypted immediately and thus are stored in the computer system only in an encrypted state. The computer system thus does not contain any unencrypted information, whereby the so-called zero-knowledge proof concept is ensured. With this concept, it is thus ensured that the provider or administrator of the computer system is himself not able to view the stored data of the users.

In addition, this procedure has the advantage that the patient data can be stored largely safely even when distributed over various sites and/or over different computers or servers and can even be kept in so-called cloud storage without any security risk.

A preferred embodiment of the method further comprises the steps: defining user permissions to process stored patient data in the user key, and processing stored patient data in the computer system by a user in dependence on user permissions to process stored patient data defined in the user's user key. The user key thus not only contains the encryption information of the study key, but also defines as such user permissions by means of which access to patient data in the computer system is controlled. This allocation of permissions can in turn itself be encrypted, in particular by means of the study key, so that manipulation of this permission allocation by unauthorized persons is likewise largely ruled out.

When defining the user permissions to process stored patient data, it is particularly preferably specified that each user may always process the data that he himself has inputted. Thus, access to the user's “own” data can very easily be ensured.

The computer-implemented method preferably further comprises the steps: defining user permissions to retrieve stored patient data in the user key, and retrieving stored patient data in the computer system by a user in dependence on user permissions to retrieve stored patient data defined in the user's user key. In a similar manner, when defining the user permissions to retrieve stored patient data, it is particularly advantageously specified that each user may always retrieve the data that he himself has inputted.

The computer-implemented method can additionally be made particularly secure in that the stored patient data are stored in the computer system in a fragmented manner.

Furthermore, with the procedure, the stored patient data can preferably also be stored in cloud storage of the computer system.

The disclosure is also directed to such a computer-implemented method having the following step: processing patient data stored in the computer system to result data, wherein the result data are stored in the computer system in an encrypted manner by means of the study key and/or by means of the respective user key. The two-step encryption, which is nested within itself, is thus preferably also used for the processing of patient data to result data and for the storage thereof.

In addition, the disclosure advantageously relates to such a computer-implemented method, wherein changes to patient data and/or result data are recorded by means of a signature using the blockchain principle. Changes to data can thus be tracked in a tamper-proof manner.

Finally, the disclosure is also directed to a computer system which is adapted to carry out such a computer-implemented method according to the disclosure.

### BRIEF DESCRIPTION OF THE DRAWINGS

An exemplary embodiment of a solution according to the disclosure will be explained in greater detail hereinbelow with reference to the accompanying schematic drawing, in which:

FIG. 1 shows an exemplary embodiment of a computer system, and

FIG. 2 shows a flow diagram of an exemplary embodiment of the method for inputting and storing patient data in a computer system.

### DETAILED DESCRIPTION

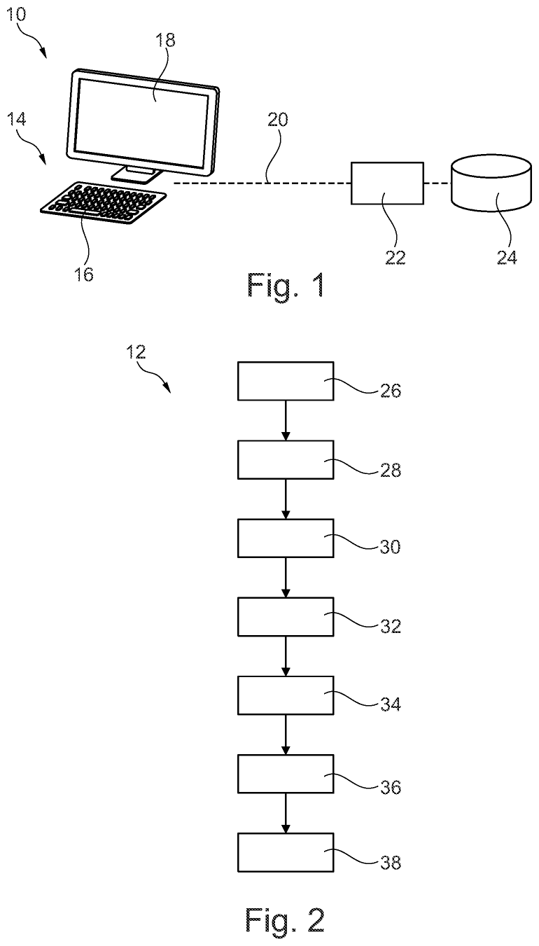

FIG. 1 shows a computer system 10 by means of which a method 12 (see FIG. 2) is to be carried out.

The computer system 10 comprises a plurality of input and output units 14, of which only one is shown by way of example. The input and output unit 14 comprises, in the manner of a terminal, at least a keyboard 16 and a screen 18. Preferably, the input and output unit 14 further comprises a computer unit (not shown in detail).

The input and output unit 14 is connected by means of a line 20, which can be wired or also wireless, to a (further) computer unit 22. A memory unit 24 is in turn operatively coupled to this computer unit 22.

By means of the computer system 10, the method 12, which is shown in FIG. 2, is to be carried out. In the method 12, in a step 26, a study key in the form of a first code is first defined by an administrator (not shown) of the computer system 10 by means of a first algorithm, which is carried out by the computer system 10, for key generation. Then, in a step 28, multiple user keys for multiple users (not shown) are defined by the administrator by means of the computer system 10, likewise by means of a second algorithm. When defining the user keys, the previously generated study key is taken into account within the second algorithm. In particular, the user keys are encrypted by means of the study key itself and/or the information of the study key is integrated into the user keys. The key length is in particular 128 bits, or 16 bytes, in accordance with the AES256 standard.

Then, in a step 30, these user keys, in particular in the form of QR codes, are allocated to each user personally by separate key card. In a step 32, the user is then able to log in to the computer system 10 using his input and output unit and the user key. In a step 34, the user can then input patient data and optionally also retrieve patient data. For inputting, the patient data are immediately encrypted by means of the user key in step 34 as they are inputted, and in a step 36 they are transmitted solely in encrypted form via the line 20 to the computer unit 22, or the memory unit 24. Alternatively, the patient data can also be kept in such encrypted form on the input and output unit 14 itself or on another computer or memory, in particular a cloud computer or cloud storage.

By means of the input and output unit 14, this user or a different user can in a step 38 retrieve and/or process these patient data at a different input and output unit 14. Access to the patient data is here controlled by means of the respective user key. Because the information of the study key is at the same time also contained in each user key, the patient data or associated result data inputted and encrypted by a first user can also be retrieved and thereby decrypted by a second user in step 38.

### LIST OF REFERENCE NUMERALS

10 computer system
12 method
14 input and output unit
16 keyboard
18 screen
20 line
22 computer unit
24 memory unit
26 step of preparing study key
28 step of preparing user keys
30 step of allocating user keys
32 step of logging on by user
34 step of inputting and encrypting patient data
36 step of transmitting patient data
38 step of decrypting and retrieving the patient data or result data

1.-10. (canceled)

11. A computer-implemented method (12) for inputting and storing patient data in a computer system (10) by multiple users, comprising:
defining (26) a study key,
defining (28) one user key per user by means of the study key,
allocating (30) the user keys to the users,
inputting (34) patient data by individual ones of the users by means of the respective user key, wherein the inputted patient data are stored in the computer system (10) in an encrypted manner by means of the user key.

12. The computer-implemented method according to claim 11, further comprising:
defining user permissions to process stored patient data in the user key, and
processing stored patient data in the computer system (10) by a user in dependence on user permissions to process stored patient data defined in the user's user key.

13. The computer-implemented method according to claim 12,
wherein, when defining the user permissions to process stored patient data, it is specified that each user may always process the data that the respective user has inputted.

14. The computer-implemented method according to claim 11, further comprising:
defining user permissions to retrieve (42) stored patient data in the user key, and
retrieving (42) stored patient data in the computer system (10) by a user in dependence on user permissions to retrieve (42) stored patient data defined in the user's user key.

15. The computer-implemented method according to claim 14,
wherein, when defining the user permissions to retrieve (42) stored patient data, it is specified that each user may always retrieve the data that he himself has inputted.

16. The computer-implemented method according to claim 11,
wherein the stored patient data are stored in the computer system (10) in a fragmented manner.

17. The computer-implemented method according to claim 11,
wherein the stored patient data are stored in cloud storage of the computer system (10).

18. The computer-implemented method according to claim 11, further comprising:
processing patient data stored in the computer system (10) to result data,
wherein the result data are stored in the computer system (10) in an encrypted manner by means of the study key and/or by means of the respective user key.

19. The computer-implemented method according to claim 11,
wherein changes to patient data and/or result data are recorded by means of a signature using the blockchain principle.

20. A computer system (10), adapted to carry out the computer-implemented method according to claim 11.
